(19) United States
(12) Patent Application Publication
(10) Pub. No.: US 2026/0338733 A1
(43) Pub. Date: Oct. 29, 2026

(54) ELECTRICAL CHARGE ABSORBER, CHARGE SENSOR AND MAGNETOMETER

(71) Applicant: Charles ROUAUD, Sevres (FR)

(72) Inventor: Charles ROUAUD, Sevres (FR)

(73) Assignee: LIPEO, Sevres (FR)

(21) Appl. No.: 19/118,288

(22) PCT Filed: Oct. 3, 2023
(86) PCT No.: PCT/EP2023/077332

§ 371 Date: Apr. 3, 2025

(30) Foreign Application Priority Data
Oct. 4, 2022 (FR) FR2210131

(51) Int. Cl. G01N 27/60; G01D 21/02; G01N 33/00; G01N 33/02; G01N 33/24; G01R 29/24; G01R 33/028; H01F 1/00; H05F 3/04

(52) U.S. Cl. CPC G01N 27/60; G01D 21/02; G01N 33/0098; G01N 33/02; G01N 33/24; G01R 29/24; G01R 33/028; H01F 1/0018; H05F 3/04

(57) ABSTRACT

One or more embodiments of the present invention relate to an absorber for electrical charge in the environment, the absorber including a diamagnetic element surrounded by an insulator, the insulator being an insulating envelope with respect to electromagnetic radiation.

The present invention relates to an electrical charge absorber. The present invention also relates to a charge sensor, a magnetometer including such an absorber and to a use of such a device.

### BACKGROUND OF THE INVENTION

Measuring electrical charges is important for many applications.

Nonetheless, the currently used apparatuses are unable to accurately measure low electrical charges.

Hence, there is a need for devices allowing measuring low electrical charges with a better accuracy.

### SUMMARY OF THE INVENTION

To this end, the description describes an absorber for electrical charge in the environment, the absorber including a diamagnetic element surrounded by an insulator, the insulator being an insulating envelope with respect to electromagnetic radiation.

According to particular embodiments, the absorber has one or more of the following features, considered separately or according to any technically-feasible combination:
the diamagnetic element is made of a diamagnetic material which is selected from the list consisting, without limitation, of:
silver,
pyrolytic carbon, in plate or sintered powder form, and
an organic compound, in particular sucrose, and
water.
the diamagnetic element has a cylindrical shape.
the diamagnetic element is a cylindrical bar provided with two ends, a first end being an open end and a second end closed by a paramagnetic element.

The description also describes a charge sensor, the sensor comprising an absorber as described before and an electromagnetic waveguide comprising a conduit, the conduit preventing the propagation of stationary waves, the conduit extending between two ends, one end being the diamagnetic element.

According to particular embodiments, the charge sensor has one or more of the following features, considered separately or according to any technically-feasible combination:
the other end of the conduit is a reflective dish.
the sensor further comprising a removable plug, the plug being made of a paramagnetic material, for example aluminium.

The description also relates to a magnetometer comprising:
an electrode rotatably mounted about an axis, the electrode being a hemispherical electrode made of a diamagnetic material,
sensor as described before, the sensor being mounted movable on a slide arranged between the sensor and the electrode,
a measuring subassembly capable of measuring the distance between electrodes upon deflection of the electrode caused by a movement of the sensor, the value of the magnetic field measured by the magnetometer being the measurement distance, and
a discharge subassembly capable of discharging the electrical charge of the electrode when a portion of the discharge subassembly is in contact with the electrode.

According to particular embodiments, the magnetometer has one or more of the following features, considered separately or according to any technically-feasible combination:
the hemispherical electrode is a black body.
the subassembly includes an electrically-conductive element made of a ferromagnetic material to perform the discharge.
the discharge subassembly includes an insulating enclosure made of a paramagnetic material.
the measuring subassembly comprises a light sensor.
the magnetometer further includes a mast to which the electrode is connected, and a device for holding the direction of the mast.
the magnetometer further includes an arm connecting the electrode to the mast, the arm being made of a paramagnetic material.

The description also describes a use of a magnetometer as described before, the charges resulting in particular from a natural induction produced by a living organism, in one of the following applications:
measurement of the diamagnetic charge of at least one portion of a living organism, such as a human, a plant or an organic compound, like an essential oil,
measurement of the diamagnetic charge of an ore, for example an ore in the way of a stony soil,
monitoring of the germination of a seed,
storage and transfer of diamagnetic energy, for a later therapeutic use, for example as skin care or fire barrier.
determination of the degree of freshness of a foodstuff, such as a fruit, a vegetable, a meat or a fish,
measurement of the charge remanence of a living organism subjected to an electromagnetic radiation, in particular an electromagnetic radiation originating from a mobile terminal,
detection of the diamagnetic gradients of a soil, and
mapping of the diamagnetic gradients of a soil, signatures of present or disappeared archaeological deposits, such as feudal mottes, arrow slits, staircases, ancient ways, protohistoric huts.

In the present description, the expression ‘capable of’ indiscriminately means ‘suitable for’, ‘suited to’ or ‘configured for’.

### BRIEF DESCRIPTION OF THE DRAWINGS

Features and advantages of the invention will become apparent upon reading the following description, given only as a non-limiting example, and made with reference to the appended drawings, wherein:

FIG. 1 is a schematic representation of an electrical charge absorber,

FIG. 2 is a schematic representation of an electrical charge sensor,

FIG. 3 is a schematic representation of a magnetometer, and

FIG. 4 is a schematic representation of a portion of the magnetometer of FIG. 3.

### DETAILED DESCRIPTION OF PREFERRED EMBODIMENTS

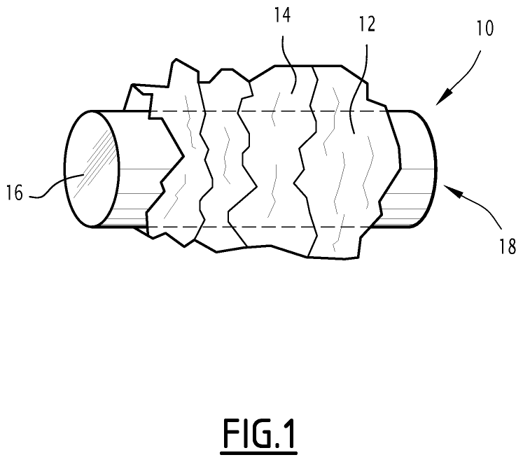

An absorber 10 is illustrated in FIG. 1.

The absorber 10 is herein an electrical-type absorber, i.e. an absorber for electrical charge in the environment.

The absorber 10 includes a diamagnetic element 12 surrounded at least partially by an electromagnetic radiation insulator 14.

As its name suggests, a diamagnetic element 12 includes a portion made of a diamagnetic material.

Diamagnetism is a behaviour of the materials which causes them, when they are subjected to a magnetic field, to create a very low magnetisation opposite to the external field, and therefore to generate a magnetic field opposite to the external field.

Several materials may be considered for the diamagnetic material.

For example, it is possible to consider silver 935 or an organic compound, such as sucrose.

According to still another embodiment, the diamagnetic material is water.

In this case, it is a silver wire having undergone a machining by cutting and polishing the ends.

Nevertheless, preferably, when a greater compactness is desired, the selected diamagnetic material is pyrolytic carbon which has the advantage of having a relatively high diamagnetic susceptibility, the latter amounting to —40.10⁻⁵. This corresponds to a size reduction of 20 in comparison with the case where the selected diamagnetic material is silver.

Pyrolytic carbon (or pyrocarbon) is a synthetic material (inexistent in nature) similar to graphite, but with covalent bonds between its graphene layers due to imperfections during production thereof. In general, it is produced by heating a hydrocarbon almost at its decomposition temperature (thermolysis), and by enabling the graphite to crystallise (pyrolysis). One method consists in heating synthetic fibres under vacuum. Another method consists in placing seeds in the very hot gas in order to cover them with a graphite deposit.

According to the described example, the diamagnetic element 12 is a solid cylinder made of a diamagnetic material.

The cylinder has a height comprised between 3 millimetres (mm) and 20 mm.

According to the described example, the height is equal to 10 mm.

The cylinder also has a base shape, which is a disk in the case of FIG. 1.

the disk has a diameter comprised between 1 mm and 10 mm.

According to the proposed example, the diameter is equal to 2 mm.

The charge and discharge time constant is proportional to the mass of the cylinder.

Thus, the diamagnetic element 12 is a cylindrical bar provided with two ends 16 and 18.

The first end 16 is an open end and the second end 18 is closed by a paramagnetic element not shown in FIG. 1.

In magnetism, paramagnetism refers to the behaviour of a material medium that does not have a spontaneous magnetisation but which, under the effect of an external magnetic field, acquires a magnetisation oriented in the same direction as the applied magnetic field. A paramagnetic material has a magnetic susceptibility with a positive value.

The insulator 14 is intended to substantially attenuate external electromagnetic radiations (such as visible or infrared light rays) to ensure that the diamagnetic element 12 could absorb the electrical charge of the immediate environment of the absorber 10.

One could herein note that the insulator 14 is thus intended to modify the radiation pattern which depends on the solid angle of aperture.

The insulator 14 is herein an insulating envelope with respect to electromagnetic radiation.

According to the illustrated example, the insulator 14 is made of aluminium.

In particular, the insulator 14 is herein a flexible aluminium film in contact with the diamagnetic element 12.

Alternatively, in the case of FIG. 1, the insulator 14 is a hollow cylinder surrounding the diamagnetic element 12.

Thus, the described absorber 10 is an absorber capable of effectively absorbing the electrical charge of the environment.

The absorber 10 could be viewed as a diamagnetic mass probe capable of acquiring and memorising a potential. Thus, the diamagnetic element 12 will keep in memory the charge sensed by the environment.

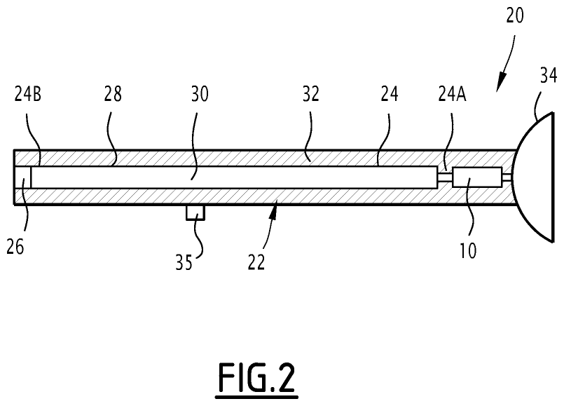

This capability can be advantageously used to acquire or measure the electrical charge. For this purpose, it is described hereafter a sensor 20 for the charge of electromagnetic energy as described with reference to FIG. 2.

The charge sensor 20 includes an absorber 10 and an electromagnetic waveguide 22.

Because of the nature of the absorber 10, the sensor 20 is a charge sensor for low charges.

The electromagnetic waveguide 22 comprises a conduit 24 and a plug 26.

The conduit 24 has a curved profile without any straight elbow in order to avoid the propagation of stationary waves.

The conduit 24 has walls 28 made of an insulating material.

For example, the insulating material is poly (ethylene terephthalate). Most often, such a material is denoted by the abbreviation PET.

Alternatively, the insulating material is a resin, in particular a resin suitable for additive manufacturing or plastics moulding.

According to the described example, the conduit 24 has the same sectional shape, which is like a ring for which an internal diameter and an external diameter could be defined.

The conduit 24 is wound around a cylinder 30.

The internal diameter is comprised between 1 mm and 3 mm, preferably equal to 2 mm.

The external diameter is comprised between 3 mm and 5 mm, preferably equal to 4 mm.

Since the diameters are relatively small, the conduit 24 could be described as a capillary.

The length of the conduit 24 is comprised between 10 cm and 100 m.

Furthermore, the conduit 24 is free of elbows having a radius of curvature greater than or equal to 10 mm.

The conduit 24 extends between two ends 24A and 24B, one end 24A being the diamagnetic element 12 and the other end 24B being the plug 26.

The plug 26 allows closing, or not, the conduit 24, the plug 26 allowing insulating the sensor 20 from the outside.

The plug 26 is removable.

The plug 26 is made of a paramagnetic material, for example like aluminium.

The sensor 20 further includes a protective case 32 surrounding the absorber 10, the conduit 24 and the plug 26.

The sensor 20 is further provided with a dish 34.

The dish 34 is a reflective dish.

The sensor 20 also comprises an index 35 allowing locating the position of the sensor 20.

One could herein note that the sensor 20 has a relatively low mass, the mass being less than or equal to 2 grams.

In operation, the sensor 20 is thus designed to be charged in less than 10 milliseconds and to memorise a charge without any alteration by conduction. In particular, the sensor 20 is insensitive to the external environment and quite particularly to radiations oriented mainly according to the main axis of the sensor 20.

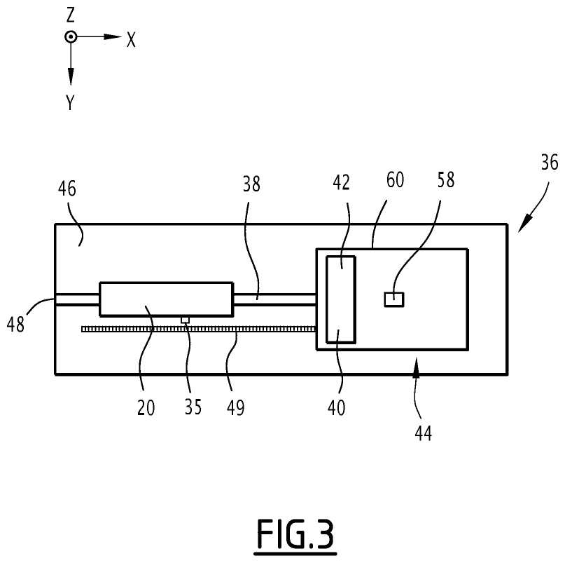

Advantageously, this allows determining a low-amplitude charge with a relatively good accuracy, for example with a magnetometer 36 as described with reference to FIG. 3.

The magnetometer 36 is an apparatus for the electrical charge in a body.

Thus, the magnetometer 36 is capable of measuring the value of the magnetic field generated by a body.

The magnetometer 36 includes the previously-described sensor 20, a movement subassembly 38, a sensing subassembly 40, a measuring subassembly 42, and a discharge subassembly 44.

All of the elements of the magnetometer 36 rest on a support 46 for which an end 48 could be defined.

The support 46 extends perpendicular to the vertical direction which is denoted by a Z axis in FIG. 3.

Thus, the support 46 extends in a plane for which a first transverse direction (denoted by an X axis in FIG. 3) and a second transverse direction (denoted by a Y axis in FIG. 3) could be defined. The X and Y axes are selected so as to form a direct orthonormal reference frame.

In the following description, to facilitate understanding, the first transverse direction will b referred to as the first transverse direction X, the second transverse direction will be referred to as the second transverse direction Y and the vertical direction will be referred to as the vertical direction Z.

The drive subassembly 38 is a slide 38.

The slide 38 is arranged between the end 48 of the support 46 and the sensing subassembly 40.

The slide 38 extends along the first transverse direction X.

The sensor 20 is mounted movable on the slide 38.

Thus, the slide 38 is arranged so as to drive the sensor 20 to the sensing subassembly 40.

For this purpose, the sensor 20 can be manually driven by an operator.

Alternatively, the sensor 20 is driven by a motor-driven plate. The motor used to move the sensor 20 is a stepper motor, for example equipped with an encoder with 200 steps per revolution.

To locate the position of the sensor 20 along the first transverse direction the slide 28 includes a graduated ruler 49 which interacts with the index 35 to obtain the pursued position.

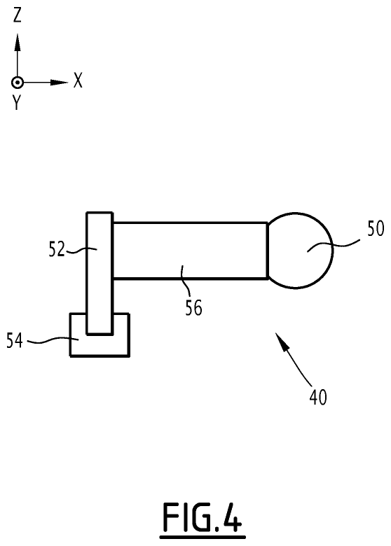

The sensing subassembly 40 is shown more specifically in FIG. 4 in the standby position.

The sensing subassembly 40 includes an electrode 50, a mast 52, a holding device 54 and an arm 56.

The electrode 50 is a hemispherical electrode made of a diamagnetic material 12.

In a specific embodiment, the electrode 50 is a hollow half-sphere with a diameter of 30 mm and has a wall of 0.4 mm.

The electrode 50 is made of a high-absorbent material, for example a black resin.

The electrode 50 herein forms a black body.

the electrode 50 is rotatably mounted about the mast 52.

The mast 52 extends according to a main direction, so-called direction of the mast 52.

As shown in FIG. 4, the direction of the mast 52 is the vertical direction Z.

The holding device 54 is capable of maintaining the direction of the mast 52. For example, the holding device 54 is a calliper 54.

For example, the calliper 54 is secured to the support 46 to guarantee a good stability of the mast 52.

Thus, the electrode 50 is rotatably mounted about the vertical axis Z.

The arm 56 connects the electrode 50 to the mast 52.

the arm 56 extends in the standby position according to the second transverse direction Y.

The arm 56 is made of a paramagnetic material, like silver.

According to a specific example, the arm 56 is a foil having a length of 70 mm. The length is the dimension according to the second transverse direction Y.

As shown in FIG. 4, the foil has a width smaller than the diameter of the electrode 50, typically 25 mm.

Such an example of a sensing subassembly 40 enables the movement of the sensor 20 generating a movement of the electrode 50. Thus, the sensing subassembly 40 somehow sense the charge stored by the sensor 20.

Thus, the movement performed by the electrode 50 is a circle portion around the vertical direction Z.

The circle portion is limited to an angle of 60°, so that the deflection of the electrode 50 is comprised between 0° and 60.

The measuring subassembly 42 is capable of measuring the deflection of the electrode 50 caused by a movement of the sensor 20, the value of the magnetic field depending on this deflection.

The law relating the value of the magnetic field and the deflection is not linear and has been determined beforehand for the magnetometer 36.

This law has a parabolic shape.

According to the described specific example, the measuring subassembly 42 includes a photovoltaic cell capable of detecting when the deflection of the electrode 50 caused by a movement of the sensor 20 exceeds a predefined threshold, for example equal to 45°.

Alternatively, according to another example, the measuring subassembly 42 includes a light sensor.

The photovoltaic cell is capable of obtaining the position of the arm 56 by determining the position where light originating from a light plane is occulted by the arm 56.

Knowing the position of the arm 56 allows knowing its angular deflection which corresponds to the angular deflection of the electrode 50.

The value of the magnetic field measured by the magnetometer 36 is related to the deflection measured by the aforementioned law.

The discharge subassembly 44 is capable of discharging the electrical charge of the electrode 50.

Such discharge takes place when a portion of the discharge subassembly 44 is in contact with the electrode 50.

In the described example, the discharge subassembly 44 includes a conductive element 58.

The conductive element 58 is an electrically-conductive element made of a ferromagnetic material allowing sensing the charge of the electrode 50.

The discharge subassembly 44 also includes an enclosure 60 which is an insulating enclosure made of a paramagnetic material.

The enclosure 60 also serves as the sensing subassembly 48 since the enclosure 60 acts as a screen by avoiding the electromagnetic disturbances originating from the outside.

Such a magnetometer 36 enables a determination of a low-amplitude charge with a relatively good accuracy.

The magnetometer 36 that has just been described can be advantageously used for many applications.

In particular, the magnetometer 36 can be used to measure electro-biological quantities, i.e. electromagnetic fields emitted by a living organism in a space, such as an animal, a plant or a bacterium, fields whose amplitude is eminently low. In particular, the electric field, the charge or the impedance can be determined.

Thus, as a particular example, the magnetometer 30 can be used to measure the diamagnetic charge of at least one portion of a living organism, such as a human, a plant or an organic compound, like an essential oil.

Thanks to this capability, it is possible to consider using the magnetometer 30 for any application based on the knowledge of an electro-biological quantity.

In particular, mention may be made of monitoring of the germination of a seed.

As another example, mention may be made of the determination of the degree of freshness of a foodstuff, such as a fruit, a vegetable, a meat or a fish.

According to still another example, mention may be made of the measurement of the charge remanence of a living organism subjected to an electromagnetic radiation, in particular an electromagnetic radiation originating from a mobile terminal.

As another example, the devices can also be used for the detection of the diamagnetic gradients of a soil. Therefore, the magnetometer 36 can be used to search for present or disappeared archaeological deposits that have left their footprint by a diamagnetic residual charge and for ancient ways since the cosmic radiations have a tendency to diamagnetise the soil. This diamagnetisation loses its homogeneity when humans build constructions.

One could herein note that the sensor is also operational in an urban environment. This sensor does not produce any artifact when passing over a sewer cover made of cast iron, or between parked cars.

According to still another example, these devices can also be used in alternative medicine.

In fact, these devices allow carrying out storage and transfer of diamagnetic energy, in particular as skin care or fire barrier.

In other words, such a device could produce a therapeutic effect equivalent to that of a magnetiser.

Indeed, the magnetiser has a diamagnetic charge concentrated in the palms of his/her hands. By performing movements close to an affected area of a patient, a charge transfer from the magnetiser to the patient takes place. Currently, the magnetiser exerts in the presence of the patient in his/her office.

With the described devices, the charge transfer would be via a buffer reservoir, for a deferred care. It could also be considered that the extent of the transfer area is controlled by using a dish with a controllable focal length.

1. An absorber for electrical charge in an environment, the absorber including a diamagnetic element surrounded by an insulator, the insulator being an insulating envelope with respect to electromagnetic radiation.

2. The absorber according to claim 1, wherein the diamagnetic element is made of a diamagnetic material selected from the list consisting of:
silver,
pyrolytic carbon,
an organic compound, and
water.

3. The absorber according to claim 1, wherein the envelope is made of aluminium.

4. The absorber according to claim 1, wherein the diamagnetic element is a cylindrical bar provided with two ends, a first end being an open end and a second end closed by a paramagnetic element.

5. A charge sensor, the sensor comprising:
an absorber according to claim 4, and
an electromagnetic waveguide comprising a conduit, the conduit preventing the propagation of stationary waves, the conduit extending between two ends, one end being connected to the diamagnetic element and the other end to a removable plug.

6. The sensor according to claim 5, wherein the plug is made of a paramagnetic material.

7. A magnetometer comprising:
an electrode rotatably mounted about an axis, the electrode being a hemispherical electrode made of a diamagnetic material, the electrode,
a sensor according to claim 1, the sensor being moveably mounted on a slide arranged so as to drive the sensor to the electrode,
a measuring subassembly capable of measuring the deflection of the electrode caused by a movement of the sensor, a value of a magnetic field measured by the magnetometer being proportional to the measured deflection, and
a discharge subassembly capable of discharging the electrical charge of the electrode when a portion of the discharge subassembly is in contact with the electrode.

8. The magnetometer according to claim 7, wherein the discharge subassembly includes at least one element selected from among:
a conductive element, the conductive element being a ferromagnetic material, and
an enclosure, the enclosure being an insulating enclosure made of a paramagnetic material.

9. The magnetometer according to claim 7, wherein the magnetometer further includes:
a mast to which the electrode is connected, and
a device for holding the mast.

10. (canceled)

11. A method of measuring a diamagnetic charge of at least one portion of a living organism which comprises utilizing the magnetometer according to claim 7.

12. A method of monitoring germination of a seed which comprises utilizing the magnetometer according to claim 7.

13. A method of determining a degree of freshness of a foodstuff which comprises utilizing the magnetometer according to claim 7.

14. A method of measuring a charge remanence of a living organism subjected to an electromagnetic radiation which comprises utilizing the magnetometer according to claim 7.

15. A method of detection of diamagnetic gradients of a soil which comprises utilizing the magnetometer according to claim 7.

16. The magnetometer according to claim 7, wherein the measuring subassembly includes a light sensor.

17. The magnetometer according to claim 9, further comprising:
an arm connecting the electrode to the mast.

18. The magnetometer according to claim 17, wherein the arm is made of a paramagnetic material.
